(19) United States
(12) Patent Application Publication
(10) Pub. No.: US 2026/0155252 A1
(43) Pub. Date: Jun. 4, 2026

(54) BIOLOGICAL AND CHEMICAL THREAT PREDICTION AND REASONING SYSTEM

(71) Applicant: A10 Systems Inc d/b/a AiRANACULUS, Lowell, MA (US)

(72) Inventors: Kakali BANDYOPADHYAY, Lowell, MA (US); Daniel GIGER, Lowell, MA (US); Zarius Ness SHROFF, Lowell, MA (US); Bryan CROMPTON, Lowell, MA (US); Apurva MODY, Lowell, MA (US)

(21) Appl. No.: 19/406,879

(22) Filed: Dec. 2, 2025

Related U.S. Application Data
(60) Provisional application No. 63/726,880, filed on Dec. 2, 2024.

(51) Int. Cl. G16H 50/20; G16H 40/67; G16H 50/30; G16H 50/70; G16H 50/80

(52) U.S. Cl. CPC G16H 50/20; G16H 40/67; G16H 50/30; G16H 50/70; G16H 50/80

(57) ABSTRACT

Aspects of the present disclosure are directed to a Biological and Chemical Threat Prediction and Reasoning System (BiCEPS). BiCEPS can leverage Artificial Intelligence (AI) and Machine Learning (ML) approaches on uniquely pre-processed data derived from wearable devices to enable early detection, monitoring, and possibly prevention (or containment) of communicable diseases that can result in pandemics. Such communicable diseases may hereinafter be referred to as biological and/or chemical threats or simply threats.

### CROSS-REFERENCE TO RELATED APPLICATIONS

This application claims priority to U.S. Provisional Application No. 63/726,880 filed on Dec. 2, 2024, the entire content of which is incorporated herein by reference.

### STATEMENT REGARDING FEDERALLY SPONSORED RESEARCH OR DEVELOPMENT

This application was made with government support under Contracts No. W81XWH-15-9-0001 awarded by the U.S. Army Medical Research and Development Command. The U.S. Government has certain rights in this application.

### BACKGROUND

With the outbreak of COVID-19 worldwide in recent years, the scientific communities around the globe as well as government officials have renewed interest in identifying and deploying mechanisms that enable early detection, monitoring, and possibly prevention (or containment) of communicable diseases that can result in pandemics.

Technological advancements have resulted in various types of wearable sensors that can play a key role in medical data acquisition. The application of wireless communication in wearable medical sensors, devices, have successfully been used to detect and monitor vital signs, vascular infarction, respiratory intensity, body temperature, blood oxygen concentration, and sleep detection, and emphasizes the key role wearable medical sensors play in acquisition and analyses of human physiological data.

Key challenges remain in determining the best approach to use data collected by such wearable devices to enable early detection, monitoring, and possibly prevention (or containment) of communicable diseases that can result in pandemics.

### BRIEF SUMMARY

Aspects of the present disclosure are directed to a Biological and Chemical Threat Prediction and Reasoning System (BiCEPS). BiCEPS can leverage Artificial Intelligence (AI) and Machine Learning (ML) approaches on uniquely pre-processed data derived from wearable devices to enable early detection, monitoring, and possibly prevention (or containment) of communicable diseases that can result in pandemics. Such communicable diseases may hereinafter be referred to as biological and/or chemical threats or simply threats.

BiCEPS provides efficient algorithms and predictive models with (a) High degree of confidence to identify threat exposure at lower than a 15% False Positive Rate (FPR); (b) Capability to provide earlier prediction before symptom onset and (c) Ability to differentiate and account for confounding factors.

In one aspect, a method includes collecting health-related data from a plurality of devices; and performing feature extraction on the health-related data. The feature extraction includes identifying statistical and temporal features of the health-related data; applying linear regression to the statistical and temporal features to determine one or more trends in the statistical and temporal features; applying one or more rolling windows to the one or more trends to identify short-term and long-term variations in the statistical and temporal features; and generating lagged values of the health-related data to determine past behavior of the statistical and temporal features. The method further includes training one or more machine learning models using the statistical and temporal features, the one or more trends, the short-term and long-term variations, and the past behavior of the statistical and temporal features.

identifying statistical and temporal features of the health-related data; applying linear regression to the statistical and temporal features to determine one or more trends in the statistical and temporal features; applying one or more rolling windows to the one or more trends to identify short-term and long-term variations in the health-related data; and generating lagged values of the health-related data to determine past behavior of the health-related data, The method further includes training one or more machine learning models using the statistical and temporal features, the one or more trends, the short-term and long-term variations, and the past behavior of the health-related data.

In another aspect, the statistical and temporal features include one or more of a mean, a median, a standard deviation, a minimum, a maximum, a skew, and a kurtosis.

In another aspect, the one or more trends include increasing and decreasing trends.

In another aspect, the one or more machine learning models include one or more of Gradient Boost, Extreme Gradient Boost, Random Forest, and AdaBoost.

In another aspect, the method further includes deploying the one or more machine learning models once the is complete; and performing threat detection using the one or more machine learning models.

In another aspect, the threat detection includes detecting one or more of a biological threat and a chemical threat.

In another aspect, the threat detection includes identifying exposure to one or more of the biological threat and the chemical threat before onset of associated symptoms.

In another aspect, the health-related data includes one or more of heart rate, sleep category data, skin temperature, respiration rate, UV light exposure, blood pressure (systolic and diastolic), ECG (heart), EEG (brain), EMG (muscle), acoustic measurements (coughing, wheezing, heart sounds), blood oxygenation, blood glucose, biomechanical measurements (e.g., via 3-axis accelerometers).

In another aspect, the health-related data is collected for a plurality of users.

In another aspect, the plurality of devices are wearable devices.

In one aspect, a device includes one or more memories having computer-readable instructions stored therein, and one or more processors. The one or more processors are configured to execute the computer-readable instructions to collect health-related data from a plurality of devices; and perform feature extraction on the health-related data by subjecting the health-related data. The feature extraction includes identifying statistical and temporal features of the health-related data; applying linear regression to the statistical and temporal features to determine one or more trends in the statistical and temporal features; applying one or more rolling windows to the one or more trends to identify short-term and long-term variations in the health-related data; and generating lagged values of the health-related data to determine past behavior of the health-related data, The one or more processors are further configured to execute the computer-readable instructions to train one or more machine learning models using the statistical and temporal features, the one or more trends, the short-term and long-term variations, and the past behavior of the health-related data.

In one aspect, one or more non-transitory computer-readable media includes computer-readable instructions, which when executed by one or more processors, cause the one or more processors to collect health-related data from a plurality of devices; and perform feature extraction on the health-related data by subjecting the health-related data. The feature extraction includes identifying statistical and temporal features of the health-related data; applying linear regression to the statistical and temporal features to determine one or more trends in the statistical and temporal features; applying one or more rolling windows to the one or more trends to identify short-term and long-term variations in the health-related data; and generating lagged values of the health-related data to determine past behavior of the health-related data, The one or more processors are further configured to execute the computer-readable instructions to train one or more machine learning models using the statistical and temporal features, the one or more trends, the short-term and long-term variations, and the past behavior of the health-related data.

In one aspect, a method includes generating a customized convolutional neural network model; collecting health-related data from a plurality of devices; and training the customized convolutional neural network model using the health-related data.

In another aspect, the customized convolutional neural network model is a 1-dimensional neural network with 1-dimensional convolutional kernels.

In another aspect, convolutional layers of the customized convolutional neural network model are followed by 1-dimensional max pooling layers.

In another aspect, the 1-dimensional max pooling layers are down sampled by a factor of 4 in one dimension.

In another aspect, after each of the 1-dimensional max pooling layers, a number of channels for the convolutional layers is doubled.

In another aspect, the convolutional layers exclude a last convolutional layer of the customized convolutional neural network model.

In another aspect, the last convolutional layer is followed by an average pooling layer.

In another aspect, the customized convolutional neural network model is trained using AdamW optimizer with a learning rate of 0.001 and a weight decay of 0.01.

In another aspect, the customized convolutional neural network model is trained using PyTorch mixed precision training.

In another aspect, a first convolutional layer of the convolutional layers uses a stride of 5 to increase a size of a receptive field for identifying long-range patterns in the health-related data.

In one aspect, a device includes one or more memories having computer-readable instructions stored therein, and one or more processors. The one or more processors are configured to execute the computer-readable instructions to generate a customized convolutional neural network model; collect health-related data from a plurality of devices; and train the customized convolutional neural network model using the health-related data.

In one aspect, one or more non-transitory computer-readable media includes computer-readable instructions, which when executed by one or more processors, cause the one or more processors to generate a customized convolutional neural network model; collect health-related data from a plurality of devices; and train the customized convolutional neural network model using the health-related data.

### BRIEF DESCRIPTION OF SEVERAL VIEWS OF THE DRAWINGS

To easily identify the discussion of any particular element or act, the most significant digit or digits in a reference number refer to the figure number in which that element is first introduced.

FIG. 1 illustrates an example system for BiCEPS according to some aspects of the present disclosure.

FIG. 2 illustrates an example software architecture for supervised training of neural network models for threat detection according to some aspects of the present disclosure.

FIG. 3 illustrates example multimodal fusion techniques according to some aspects of the present disclosure.

FIG. 4 is a table illustrating the results of threat detection using ensemble techniques according to some aspects of the present disclosure.

FIG. 5 illustrates a method of training a machine learning model using ensemble techniques for threat detection according to some aspects of the present disclosure.

FIG. 6 illustrates a method of developing and training a convolutional neural network for threat detection according to some aspects of the present disclosure.

FIG. 7 illustrates a method of threat detection using one or more trained machine learning models according to some aspects of the present disclosure.

FIG. 8 shows an example of computing system that can be used as any one or more components of BiCEPs according to some aspects of the present disclosure.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and, such references mean at least one of the embodiments.

Reference to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

Without intent to limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, technical and scientific terms used herein have the meaning as commonly understood by one of ordinary skill in the art to which this disclosure pertains. In the case of conflict, the present document, including definitions will control.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

In recent years, health data analyses approaches, based on intelligent techniques like AI and ML have been increasingly used for leveraging data, derived from wearables/sensors, to generate knowledge and information. With the outbreak of COVID-19 worldwide, wearable medical sensors have been playing a key role in medical data acquisition. The application of wireless communication in wearable medical sensors, devices, have successfully been used to detect and monitor vital signs, vascular infarction, respiratory intensity, body temperature, blood oxygen concentration, and sleep detection, and emphasizes the key role wearable medical sensors play in acquisition and analyses of human physiological data. Majority of the wearable devices can record and monitor body temperature, respiratory rate, heart rate, blood pressure as the most frequent vital signs. These can be used for early detection, monitoring, and prevention of the spread of communicable diseases and early detection of any outbreak or bio threat agent. Specialized wearables used in health care domains may measure parameters including, heart rate, skin temperature, breathing rate, UV light exposure, blood pressure (systolic and diastolic), ECG (heart), EEG (brain), EMG (muscle), acoustic measurements (coughing, wheezing, heart sounds), blood oxygenation, blood glucose, biomechanical measurements (e.g., via 3-axis accelerometers). However, key challenges remain in feature selection to allow for right identification of causative agents for the diseases, due to overlapping initial physiological symptoms exhibited by patients for diseases caused by different infectious agents including bio and chemical threat agents. There needs to be focused efforts to identify and develop AI/ML models that would enable (a) Identification of the threat agent (b) Earlier prediction of threat exposure before symptom onset, with high confidence and (c) Ability to differentiate and account for confounding factors.

As will be described in more detail below, aspects of the present disclosure provide software tools with capability to predict threat exposure based on analysis of data derived from wearable devices. Such software tools may be provided in any language such as Python or an equivalent high level programming language, which can be stored on one or more memories and executable by one or more processors to implement the functionalities included in the tools for threat detection and exposure prediction. The software tools further provide AI/ML based models based on exploration of the use of a variety of AI/ML techniques, including (but not limited to) Deep Convolutional Neural Networks (DCNN), Ensemble techniques, Extreme Gradient Boost, Recurrent Neural Networks etc., that work well with symptomatic and asymptomatic patient derived samples, representing realistic field conditions, and can predict threat exposure before symptom onset.

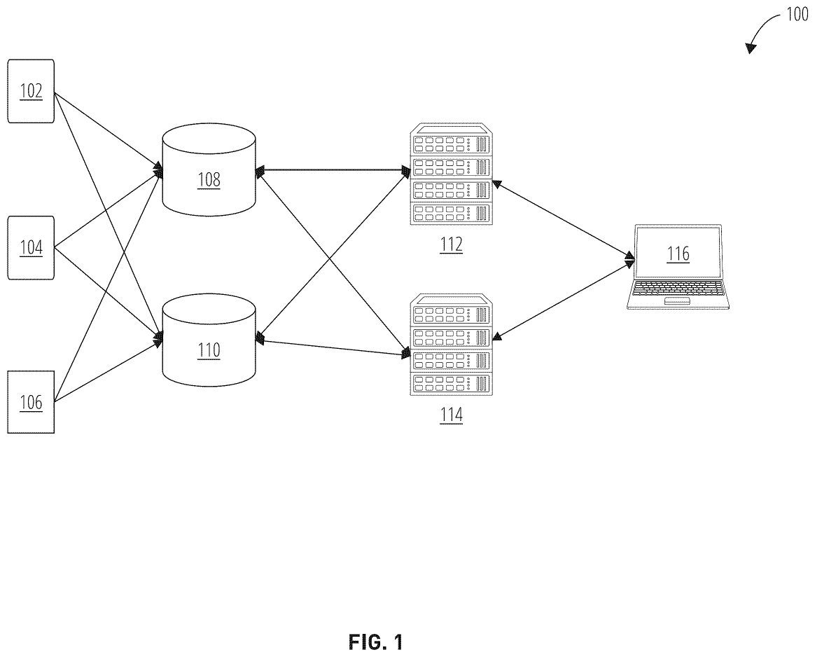

Prior to describing various aspects related to developed AI/ML mechanism and software tool for prediction of threats, a non-limiting example of a system for implementing BiCEPS will be described with reference to FIG. 1.

FIG. 1 illustrates an example system for BiCEPS according to some aspects of the present disclosure.

Example architecture 100 is a generic illustration of BiCEPS used for gathering data from wearable devices to be stored in one or more databases for pre-processing and training one or more neural networks that can then be used for detection of threats described above. Example architecture 100 is non-limiting and for illustration purposes only. Any other known or to be developed configuration of connected network elements for gathering and processing health-related data, training neural network models, and deploying such trained models for detection of threats, may be used instead and falls within the scope of the present disclosure.

As shown in FIG. 1, architecture 100 may include a number of wearable devices, including device 102, device 104, and device 106. Each one of device 102, device 104, and device 106 may be any known or to be developed health monitoring device including, but not limited to, commercially available wearable devices such as Garmin® watches, Empatica® watches, Oura® ring, Apple® watches. While FIG. 1 shows three wearable devices as examples for illustrative purposes only, the number of wearable devices are not limited to those shown as part of architecture 100. In practice, health data may be collected from hundreds, thousands, or millions of wearable devices.

Health data collected by device 102, device 104, and/or device 106 may be communicated (via wired and/or wireless communication) to one or more databases such as database 108 and database 110. This transmission of health data may be periodic or in real-time. The number of databases used for collecting and storing health data may be more or less than database 108 and database 110 shown in FIG. 1. Database 108 and database 110 may be remotely located relative to other components of architecture 100 or alternatively may be physically located in the same location as one or more additional components of architecture 100. In one example, database 108 and/or database 110 may be cloud-based hosted on a public, private, and/or a private-public cloud service.

Communication among various components of architecture 100 may be wired or wireless using any known or to be developed wired and/or wireless communication scheme.

Architecture 100 may further include one or more processors such as processor 112 and process 114. The number of processors is not limited to that shown in FIG. 1 and can be more or less. Processor 112 and processor 114 may be cloud-based or local.

Architecture 100 may further include one or more terminals such as laptop 116 connected to processor 112, processor 114, database 108, and/or database 110 to control one o more operations thereof. The one or more terminals may be a laptop, a desktop computer, a tablet, a mobile device, etc.

In one example, in addition to health data collected by device 102, device 104, and/or device 106, database 108 and/or database 110 may further store trained neural network models that can then be executed by processors such as processor 112 and processor 114 for real-time detection of threats.

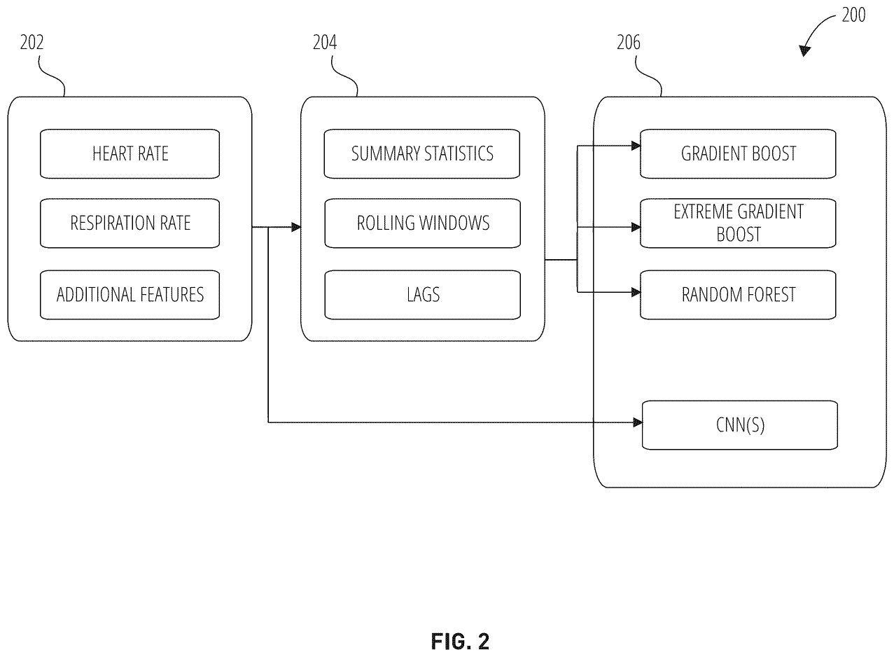

FIG. 2 illustrates an example software architecture for supervised training of neural network models for threat detection according to some aspects of the present disclosure.

Software architecture 200 of FIG. 2 may be executed by architecture 100 and more specifically by processor 112 and/or processor 114 of FIG. 1.

Software architecture 200 has three components, namely, features 202, feature extraction 204, and models 206.

Features 202 may represent various health-related data collected by devices such as device 102, device 104, and device 106. Health-related data include, but are not limited to, heart rate, respiration rate, and any additional health-related data such as those described above (e.g., sleep data).

Feature extraction 204 may include various processing techniques that may be performed on data collected by devices (wearable devices) including, but not limited to, summary statistics, application of lags, rolling windows, etc. These techniques will be further described below. As will also be described further below, these processing techniques may be applied to collected health-related data for training one or more neural networks.

As noted above, exemplary aspects of the present disclosure include unique processing techniques applied to health-related data for training one or more ensemble models. These unique processing techniques enable the high success rate of AI/ML techniques in detection of threats.

Models 206 may include one or more modes that may be subject to supervised or semi-supervised learning for threat detection. Non-limiting examples of such models may include ensemble techniques (e.g., gradient boost, extreme gradient boost, random forest, etc.) and custom-developed neural networks (e.g., CNNs). As shown in FIG. 2 and as will be described below, health-related data may be directly provided as input into custom-developed CNNs for supervised learning without being subject to processing techniques of feature extraction 204. Health-related data that is not subject to processing techniques described may be referred to as raw data (represented in time-series).

Data loading may refer to the process of loading health-related data from devices that collect such data (e.g., wearable devices such as device 102, device 104, and device 106) into databases such as database 108 and/or database 110 for pre-processing and/or training models for threat detection.

Data loading may be performed using various known or to be developed open-source libraries (e.g., open-source libraries for Python). In one example, Pandas may be used. However, relaying on Pandas alone may introduce delays. For example, loading all the data for a specific device may take up to 40 minutes. In one aspect, the present disclosure introduces PyArrow into the work pipeline for loading the data, which brought the time to load the data down to 5-10 minutes for a single device ID. PyArrow is a highly optimized library, with efficient support for the Parquet files used in datasets.

In one aspect, per-device datasets may be separated into files based on the device ID associated with each time series. This reduces the loading time down to around 0.02 seconds for the data associated with one device. Prior to this change, the data for each device was spread out through multiple Parquet files and selecting the data for a specific device required scanning through the entire dataset.

While PyArrow tends to be much more performant than the Pandas library, PyArrow is less flexible. Accordingly, the present disclosure utilizes a combination of PyArrow and Pandas when loading the health-related data. In one instance, PyArrow is used for the most performance-sensitive sections of code, while Pandas is used for the rest, where the increased flexibility is worthwhile. To do so, data from the Parquet files may be loaded and only the desired columns may be selected using PyArrow. The selected data may then be converted to Pandas.

In another aspect, the data loading pipeline may further be optimized using Python's multiprocessing module. In Python, there are various forms of concurrency available, but only some of them are well-suited for parallelization because of Python's Global Interpreter Lock (GIL). In some forms of concurrency, such as multithreading, the GIL tends to prevent more than one thread from running at the same time, which limits Python's ability to speed up computations. In Python, using multiprocessing can circumvent issues with the GIL, because each process gets its own GIL, rather than competing over one shared GIL. Accordingly, the software code for the data loading pipeline of the present disclosure is parallelized to substantially speed up data loading, which enabled faster iteration over different modeling approaches.

After parallelizing data loading code, the code was profiled using the Py-Spy sampling profiler and it was discovered that the code was spending a significant portion of time converting strings into timestamps. To eliminate this bottleneck, the timestamps on the filesystem are stored using the format which PyArrow and Pandas can use directly. This further speeds up data loading by performing the conversion once, instead of needing to convert the strings to timestamps every time a dataset is loaded.

As noted above, modeling techniques used as part of modeling 206 include two ML approaches, namely, ensemble techniques and CNN models. With reference to ensemble techniques, unique pre-processing techniques are applied to raw time-series health-related data collected via wearable devices such as device 102, device 104, and device 106. CNNs are custom-developed according to some aspects of the present disclosure and raw time-series health-related data are used for training the CNNs. Each of these techniques are described below in more detail.

### Ensemble Techniques

During the development of the ensemble models, direct usage of the raw time series data is suboptimal for training the models due to its inherent noise and high dimensionality. To address this, in some aspects, the techniques disclosed herein rely on pre-processing of the raw data (e.g., health-related time series data collected by wearable devices) for feature extraction. In one example, the raw data may be transformed into a structured set of features more amenable to the ensemble techniques used. The feature extraction process may include applying a custom function that computes statistical and temporal features from the raw data. Summary statistics such as mean, median, standard deviation, minimum, maximum, skew, and kurtosis may be determined. Next, linear regression may be used to determine the increasing and decreasing trends in the data. Next, rolling means and standard deviations may be determined over windows of various sized to capture both short-term and long-term variations. Finally, lagged values of the time series may be created to help the models understand the past behavior of the time series data. This feature extraction technique can have positive effect on both the results and the training time of the ensemble models. This is due to a multitude of reasons, namely, dimensionality and noise reduction. Reducing the dimensionality of the data speeds up the training time, while allowing the model to focus on a smaller set of relevant features. In addition, feature extraction filters out some of the noise in the minute fluctuations of the raw data and minimizes the impact of outliers. Feature extraction also enhances versatility in the models, allowing usage of techniques that would otherwise be unsuitable for raw time series data.

Ensemble techniques utilized in the present disclosure include, but are not limited to, Random Forest, Gradient Boost, Extreme Gradient Boost, and AdaBoost. All these techniques use Decision Trees as a base classifier to make predictions on. A decision tree predicts a label by traveling from a root node to a leaf. At each node on the root-to-leaf path, the successor child is chosen by splitting the input space. Boosting methods combine multiple “weak learners” into a single “strong learner” in a sequential manner. Each weak learner typically has only a slight edge over random guessing, but when combined, they achieve high accuracy. The process focuses on improving the predictions by iteratively learning from the mistakes of the ensemble of models built up to that point. Gradient Boosting builds models in a stage-wise fashion like other boosting methods but generalizes them by allowing optimization of an arbitrary differentiable loss function. The method involves sequentially adding classifiers to a model, with each addition attempting to correct the residuals left by the previous models. Extreme Gradient Boosting is an implementation of gradient boosted decision trees designed for speed and performance. It is a scalable and accurate implementation of gradient boosting with support for parallel tree boosting (also known as GBDT, GBM). AdaBoost (Adaptive Boosting) focuses on classification problems. The idea is to increase the proportional weight of previously misclassified observations. Each ensemble model has a set of configurable parameters that determine the structure and behavior of the algorithm called hyperparameters. Hyperparameters may be optimized using a technique called grid search, which iterates through all combinations of hyperparameters given in a parameter grid, and outputs the best combination. In addition to grid search, manual tuning of the models may also be performed.

As noted above, ensemble techniques rely on pre-processing techniques to extract valuable features that optimize the output of models used for threat detection. These pre-processing techniques can extract statistics from raw data as described above through determining statistical and temporal features, apply lags (e.g., 1, 5, 15, 30, 60, 1440) and rolling windows (e.g., using window sizes such as 15, 30, 60, 720, 1440, 2880) to such statistics to capture trends, and uses them in training the models.

To tune the hyperparameters for the ensemble models, a combination of grid search and manual tuning may be used. Grid search allows for trying every combination of hyperparameters within a parameter grid. Examples of hyperparameters include:
n_estimators: This is the number of decision trees used in the model. The more trees, the more complex trends the model is able to capture, but also the more likely it is to overfit to the training data. It is important to strike a balance between overfitting and underfitting.
max_depth: This is the maximum depth that each decision tree is allowed to grow to. Again, the more depth each tree has the more complex the model is, but also this can lead to overfitting.
learning_rate: This is the step size the optimizer uses when updating its weights. The smaller the learning rate the slower the model converges, however the more likely it is to be moving in the right direction. When the learning rate becomes too large, the model's performance can jump around quite a bit. Generally, it is best to find the largest learning rate that converges with monotonically decreasing loss.
subsample: This is the proportion of data used in each tree. A subsample too high can result in overfitting, while a subsample too low can result in underfitting. The most common values are between 0.5 and 1.
colsample_bytree: This is the proportion of features used in each tree. It is effectively the subsample of the columns. Again, a higher colsample_bytree value increases the complexity of the model but can lead to overfitting, and vice versa for underfitting. The most common values are again between 0.5 and 1.
min_child_weight: This is the minimum sum of weights required to create a new tree node. The smaller min_child_weight value is, the more complex the model can grow, which could lead to overfitting. A higher value leads to a more conservative model that could lead to overfitting.
scale_pos_weight: This is used to control the balance of the positive and negative class weights. It is used when there is an imbalanced dataset to be trained on as is the case in this problem. A common value to use is the ratio of negative to positive data points in the dataset.
eval_metric: This is used to monitor the performance of the model on the validation set during training. The evaluation metric is chosen based on the type of result the problem requires. Log loss was chosen because of the nature of this binary classification problem.

### Convolutional Neural Network (CNNs)

CNNs are known to be effective for classifying time series data, which is suitable for use on the data from wearable devices. While the most popular versions of CNNs tend to be two-dimensional, custom-developed CNNs are utilized in the context of the present disclosure that rely on one-dimensional convolutions. This is primarily because the one-dimensional nature of the time series data provided by wearable devices tends to make this the most suitable option among CNNs. Many of the principles developed in the extensive research into two-dimensional CNNs, such as on the ImageNet competition, can be applied to one-dimensional CNNs as well. In general, CNNs tend to use a combination of convolutional layers and downsampling layers. Modern CNNs tend to also include normalization layers, such as Batch Normalization, which will stabilize and accelerate the training, and often improve accuracy as well. The initial convolutional layers tend to learn basic shapes, while later layers built on that to learn to recognize more complex patterns. The convolutional layers towards the end of the model tend to learn patterns that are more class-specific, rather than straightforward geometric shapes. The combination of convolutional layers and downsampling layers tends to extract valuable features from the input data, which can then be effectively used by one or more fully connected layers to produce a final classification. For the kinds of data where CNNs are well-suited, this tends to be far more effective than directly using fully connected layers.

As part of creating the custom-developed CNNs of the present disclosure, many variations were considered, such as using models of different width, depth, kernel size, strides, dilations, and learning rates. Width and depth are two ways to adjust the size of a model, which can substantially affect how capable it is. Kernel sizes, strided convolutions, and dilation can control the size of the model's receptive field, which determines the upper limit of the size of patterns the model can identify. The learning rate affects the size of the updates at each gradient step, and influences whether the model underfits or overfits. Two of these factors which had impact were the kernel size and the stride length. Depending on the rate of the time series data used, in some examples, adjustments may be made to both the kernel size (for all convolutional layers) and the stride length (for the first convolutional layer only) to adjust the size of the network's receptive field. The receptive field is what determines the upper limit on the size of patterns that a CNN can identify. If the receptive field is too small, then the convolutional layers will miss larger patterns. These larger patterns appear to be important for detecting COVID from the long time-series provided by wearable devices. The different time series provided for the different features each have their own sampling rate. For example, some of the heart rate data is in 5-minute intervals, while the respiration data is in 1-minute intervals. This difference in intervals changes the size of the patterns which need to be identified and was the primary motivation for exploring techniques which adjust the CNNs' receptive field sizes. For the 1-minute intervals in the respiration data, a significantly larger receptive field was found to be much more effective than using the same receptive field which had worked well on the 5-minute intervals in the heart rate data.

### Multimodal Fusion

When dealing with multiple different features, it can be advantageous to train models which combine the features. One approach can be to combine the different features using concatenation, which is a form of early fusion. Different forms of feature fusion, such as late fusion, may be utilized in which models' outputs are combined, rather than combining the features before passing them into the model.

When joining the datasets, an important consideration is dealing with the different sampling rates between the features, even on the same device. For example, the heart rate data on Garmin watches are sampled at 15 second intervals, while the respiration rate data on the same Garmin watches are sampled at 1-minute intervals. To join these two features to the same timestamps, they need to be resampled to the same sampling rate. In this case the features were resampled to 1 minute using interpolation. Another consideration with multi-modal fusion, is the type of join when combining features. This is because the different features are often not collected at the same time even on the same device; that is, there can be gaps in the heart rate for a period of minutes or hours, where there is no gap in the respiration data, and vice-versa. Two example types of joins in consideration are inner and outer joins, as there is no principal feature for a left or right join. An outer join preserves the most data, however, it also forces data to be artificially created, whereas an inner join discards more data, but preserves its integrity. An inner join was chosen for these purposes as it produced better results after preliminary tests.

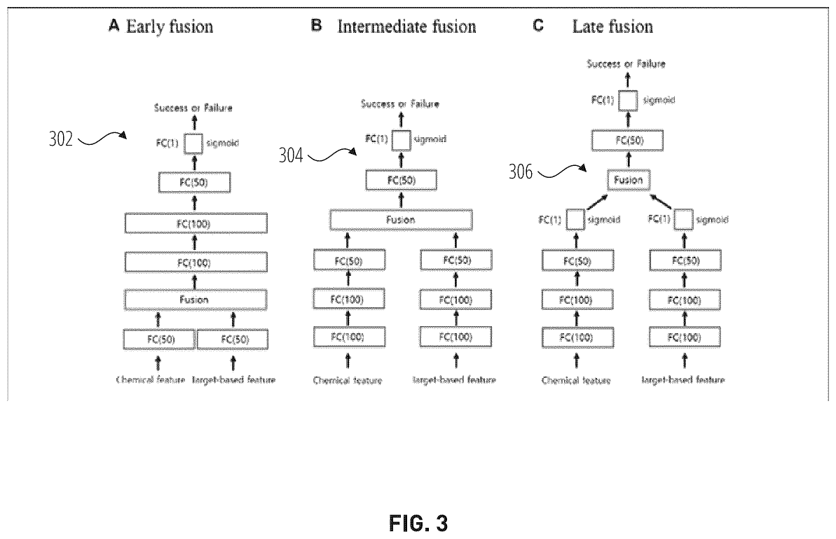

FIG. 3 illustrates example multimodal fusion techniques according to some aspects of the present disclosure.

Techniques for multimodal fusion can be roughly divided into three categories: early fusion 302, intermediate fusion 304, and late fusion 306. The difference between these approaches is when they combine the different modalities. Early fusion 302 combines the data towards the beginning of the processing. One way to do this is to concatenate the features before they are fed into the model. In one example, this approach is applied to Ensemble techniques within BiCEPS of the present disclosure.

Intermediate fusion 304 tends to combine the modalities partway through the model. One way that this can be done is if different neural network components are being used as feature extractors for different modalities. For example, it is possible to use components of a CNN, such as convolutional layers and downsampling layers to create a learned feature extractor. After passing some of the data through this feature extractor, it is possible to concatenate the extracted features with other features, and then classify the combined result using a Multilayer Perceptron at the end.

Late fusion 306 is one where the modalities are combined towards the end of the processing. One way to perform late fusion is to have entirely separate models for each modality, and combine their decisions, rather than combining the models or data directly. One effective way to combine the decisions is to average them after using a SoftMax layer. This makes it possible to incorporate the models' confidence levels, and tends to prevent issues like tied votes, even if there are an even number of models.

In some aspects of the present disclosure, for CNN models used in BiCEPS, late fusion 306 is advantageous as it illustrates significant benefits compared to early fusion 302 and intermediate fusion 304 because the models for each modality can be trained separately. This can increase flexibility and make it easy to adapt if some of the kinds of data are missing. An additional benefit for late fusion is that late fusion 306 allows use of more of the available data, which can improve the results because machine learning models are often heavily dependent on the quantity of data used to train them, especially for neural network models. In early fusion 302 approach, any sample which is missing one of the features that the models use is discarded. With a late fusion 306 approach, using separate models for each feature, there is no need to discard data that is missing one or more features. As a result of using more of the data, the trained models demonstrate more flexibility when features are missing.

### Ensemble Models Results

The initial results from the ensemble models used a data window around each participant's Covid test. These results were using multimodal fusion as described earlier on the Garmin wristwatch respiration rate and heart rate data. One of the main challenges when working with this dataset, is that it is heavily imbalanced towards the negative class because there are about 10 times more negative records than positive records. This causes models to bias towards the majority class, (negative, in this case). Some techniques used to mitigate the bias caused by the imbalanced dataset are oversampling and undersampling. Oversampling is a technique that involves increasing the number of records in the minority class (positive, in this case) by either randomly duplicating pre-existing minority records or creating artificial minority records via interpolation. One of the disadvantages of oversampling is the increased risk of overfitting to the newly increased minority class, as it is made up of repeated or closely derived records. Undersampling, on the other hand, removes some of the majority records, either randomly or by selection. The disadvantage of undersampling is the loss of potentially valuable data; however, since undersampling decreases the total number of records it can also speed up the training time, making models more efficient.

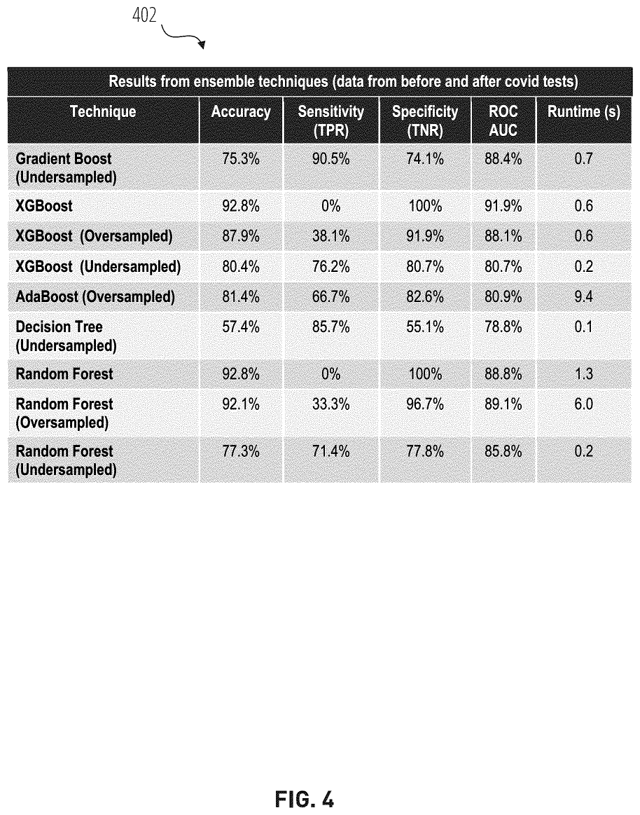

FIG. 4 is a table illustrating the results of threat detection using ensemble techniques according to some aspects of the present disclosure.

As can be seen from table 402, Sensitivity (True Positive Rate) of the models without an imbalance mitigation technique (oversampling or undersampling) is 0%. This is because in these cases the models were so biased by the class imbalance, that they always predict negative. These results suggest that the Extreme Gradient Boost Undersampled may produce the best results This model manages to maintain a high accuracy of 80.4% while also having a sensitivity of 76.4%. Although this model does not have the highest accuracy or Receiver Operating Characteristic Area Under the Curve (ROC AUC), it looks to have the best balance of metrics. In addition, to the tabular results, the models ROC curves are plotted against each other for comparison across different thresholds.

In one example, the ROC Curves of the ensemble techniques can be compared against one another. In this case, the ensemble models show very similar results, with most of them having an 80-85% True Positive Rate at a 15% False Positive Rate.

The next set of results uses a data window starting 5 days before and ending at the time of the Covid test. This is a more accurate representation of the data that will be used when predicting Covid using wearables data, as it does not include data from after the Covid test. These results use Extreme Gradient Boost as the chosen ensemble technique, as it seemed to perform best when compared to other ensembles.

In another example, a larger data window of about 14 days before the test is used, and also it uses data from symptomatic participants only. In survey data, the participants were asked to mark down whether they were experiencing any Covid symptoms at the time of their Covid tests. This allows to separate the data into three categories: symptomatic, asymptomatic, and no response, and run the models on each separately. This result also uses a time window that ends 48 hours before the Covid test. That is, wearables data from 14 days before to 48 hours before the Covid test is used. This was done to ensure models have predictive capabilities earlier in the disease lifetime, which allows for measures to be taken before further spread can occur. This approach resulted in a true positive rate of 82% at 15% false positive rate, with a ROC AUC around 87%. Another challenge with this result is a possible data leakage issue, specifically, target leakage. Because the participants may have tested repeatedly within the 14-day window, some of the data before one test may also be data after another test.

To address this, the next models used non-aggregated data for each participant. That is, each block of data is treated as separate inputs into the model. These results are less predictive than the results with aggregated data blocks. This approach resulted in a True Positive Rate of 58% at 15% False Positive Rate, with a ROC AUC around 75%. This result also uses a time window of 14 days before to 48 hours before the Covid test, and thus is our most realistic solution with the ensemble models. This result also uses asymptomatic cases only, which means this model can identify those participants who would otherwise be unable to identify themselves as sick.

In another example, Long Short-Term Memory (LSTM) models are tested. This is a type of recurrent neural network specifically designed to deal with the vanishing gradient problem with the use of a forget gate. This allows LSTM models to be especially useful when dealing with data over long periods of time. Initially, the LSTM models showed promise on a balanced dataset, scoring a 92% ROC AUC with a 65% True Positive Rate at a 15% False Positive Rate. However, the LSTMs are less effective at dealing with huge class imbalance in a dataset.

In some aspects, additional experiments with various techniques designed for time series classification are examined, which are reported in Table 1 below. ROCKET is based on randomized convolutions. MultiRocket is a variation of ROCKET designed to be faster. FCN is a Fully Convolutional Neural Network, which is unusual in that it does not use any downsampling. Arsenal is an ensemble of ROCKETs, and HIVECOTE2 is a meta-ensemble, which combines four different ensembles, including Arsenal. The results in the above table were skewed by the imbalanced dataset, and most of them can be easily adjusted for this using thresholding on their predicted values, as is done when making a ROC curve.

TABLE 1
Generic Time Series Classification Results

| | | Sensitivity | Specificity | Training Time |
|---|---|---|---|---|
| Technique | Accuracy | (TPR) | (TNR | (minutes) |
| MultiRocket | 95% | 9% | 99% | 0.8 |
| ROCKET | 95% | 6% | 99% | 1.3 |
| FCN | 95% | 0% | 100% | 38 |
| HIVECOTE2 | 95% | 3% | 100% | 90 |
| Arsenal (ensemble | 95% | 6% | 100% | 10 |
| of ROCKETs) | | | | |

### CNNs Results

In some of aspects, custom-developed CNNs on health-related data collected using wearable devices such as Oura Ring were tested. The data collected includes heart rate data, including data both before and after Covid tests. With this approach, an AUC of 0.81 can be achieved.

Some of the most promising results for symptomatic data were from the CNN models, which were able to achieve an AUC of 0.70 using respiration and heart rate data from Garmin devices. In this approach, one model was trained on the heart rate data, and another model was trained on the respiration data. The predictions of these two models were averaged and then thresholded to obtain a final decision. The thresholding was used to handle the imbalanced nature of the dataset, which tended to bias models towards predicting COVID negative. For the CNN models, thresholding was used instead of oversampling or undersampling. The results using late fusion were comparable to results using only the heart rate data. At a false positive rate of 0.11, the CNN late fusion model was able to achieve a true positive rate of 0.48.

Neural networks such as CNNs often have hyperparameters which should be configured to get good results. Many of these hyperparameters were tuned using a combination of grid search and manual tuning.

According to some aspects of the present disclosure, neural CNNs are customized to look for long range patterns and improve performance based on information known about the dataset. These customized neural networks may also be referred to as Customized neural networks for specific dataset (RAPIDS).

In one example, 1-dimensional (1D) convolutional kernels each having a kernel size of 13 were used. Most of the convolutional layers are followed by 1D max pooling layers which downsample by a factor of 4 in one dimension. After each max pooling layer, the number of channels for the convolutional layers is doubled. The first convolutional layer increases the number of channels to 10. After the last convolutional layer, there are 320 channels. The only convolutional layer which is not followed by a max pooling layer is the last one, which instead is followed by an average pooling layer instead. The CNNs were trained using the AdamW optimizer using a learning rate of 0.001 and weight decay of 0.01. PyTorch's mixed precision training was used to improve efficiency. The first convolutional layer uses a stride of 5 to increase the size of the receptive field and enable the CNNs to look for longer range patterns. Table 2 below illustrates an example comparison of the different methods, including the previous approach publicly published.

TABLE 2
Comparative Results of the different techniques

| | Approach from | | Convolutional |
|---|---|---|---|
| | previous published | Ensemble Methods | Neural Network |
| | work | Approach | Approach |
| Features | Heart Rate, | Heart Rate, | Heart Rate, |
| | Respiration Rate, | Respiration Rate, | Respiration Rate |
| | Sleep data | Sleep data | |
| Devices | Garmin and Oura Ring | Garmin | Garmin |
| | (Simultaneously only) | | |
| Time Window | 14 days prior - Covid | 14 days prior - 48 | ~23 days prior - |
| | Test | Hours before | Covid Test |
| | | Covid Test | |
| Asymptomatic/ | Symptomatic | Asymptomatic | Symptomatic |
| Symptomatic | | | |
| ROC-AUC | 81.5% | 65.6% | 70% |
| Sensitivity (TPR) | 60.5% | 37.7% | 48% |
| Specificity (TNR) | 88.8% | 85.3% | 89% |
| Accuracy | Not Available | 80.9% | 87% |
| Runtime (seconds) | Not Available | 0.2 | 10 min (approx.) |

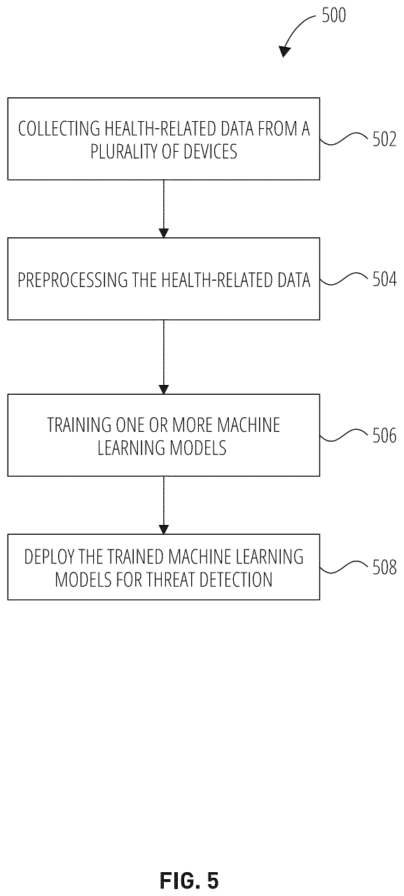

FIG. 5 illustrates a method of training a machine learning model using ensemble techniques for threat detection according to some aspects of the present disclosure.

Method 500 may be performed by architecture 100 of FIG. 1 executing software architecture 200 of FIG. 2, for example by processors 112, 114 operating on health-related data stored in databases 108, 110.

At step 502, health-related data is collected from a plurality of devices. In one example, the plurality of devices includes wearable devices such as device 102, device 104, and device 106, which may be configured to monitor and record health-related parameters for a plurality of users over time. The health-related data may include, without limitation, heart rate, respiration rate, sleep category data, skin temperature, breathing rate, UV light exposure, blood pressure (systolic and diastolic), ECG, EEG, EMG, acoustic measurements (e.g., coughing, wheezing, heart sounds), blood oxygenation, blood glucose, and biomechanical measurements (e.g., via 3-axis accelerometers). The collected health-related data may be transmitted over wired and/or wireless communication links to one or more storage resources, such as databases 108, 110, and may be organized per device and/or per user.

At step 504, the health-related data is preprocessed by performing feature extraction on the health-related data. In some aspects, feature extraction at 504 corresponds to feature extraction 204 of FIG. 2 and includes transforming raw time-series data into a structured set of statistical and temporal features that are more amenable to ensemble modeling. For example, a feature extraction module may compute summary statistics (statistical and temporal features) such as mean, median, standard deviation, minimum, maximum, skew, and kurtosis for windows of the health-related data. The feature extraction module may further apply linear regression to the statistical and temporal features to determine one or more trends in the statistical and temporal features, including increasing and decreasing trends. One or more rolling windows of various sizes (e.g., 15, 30, 60, 720, 1440, or 2880 samples) may be applied to capture short-term and long-term variations in the statistical and temporal features. Lagged values of the time-series data may additionally be generated (e.g., using lags of 1, 5, 15, 30, 60, or 1440 samples) to characterize past behavior of the statistical and temporal features and to provide temporal context to the models. These and other feature extraction operations can reduce dimensionality and noise, improve training efficiency, and expose salient temporal patterns relevant for threat detection.

At step 506, one or more machine learning models are trained using the features derived at 504 including the statistical and temporal features, the one or more trends, the short-term and long-term variations, and the past behavior of the statistical and temporal features. In some aspects, models 206 of FIG. 2 include one or more ensemble models that operate on the statistical and temporal features, the trend information, the rolling-window features representing short-term and long-term variations, and the lagged values representing past behavior. The one or more machine learning models may include one or more of Gradient Boost, Extreme Gradient Boost, Random Forest, AdaBoost, and other ensemble techniques that use decision trees as base learners. The ensemble models may be trained on labeled datasets in which positive examples correspond to instances of biological and/or chemical threat exposure and negative examples correspond to non-exposed instances. Various imbalance-mitigation strategies such as oversampling, undersampling, class-weighting, and/or threshold adjustment can be employed during training to address skew in the dataset. Hyperparameters for the ensemble models (e.g., number of estimators, maximum depth, learning rate, subsample, colsample_bytree, min_child_weight, and scale_pos_weight) may be tuned using grid search and/or manual tuning to achieve desired performance metrics such as sensitivity, specificity, and ROC AUC.

At step 508, the trained machine learning models are deployed for threat detection. For example, once training and validation are complete, the resulting ensemble models may be stored in databases 108, 110 and loaded by processors 112, 114 for on-line inference as new health-related data streams in from the plurality of devices. In some aspects, method 500 may be repeated periodically or on demand using updated health-related data to retrain and refresh the ensemble models so that threat detection performance is maintained or improved overtime. Although shown as a sequence of discrete steps, the operations of method 500 can be performed in different orders, can be combined, or can be executed in parallel, and one or more steps may be repeated or omitted in various implementations.

While Covid 19 is used as an example of a threat to be detected in describing example embodiments above, the present disclosure is not limited thereto. Any known or to be developed threat that can be detected using features extracted from health-related data, as described above, falls within the scope of the present disclosure.

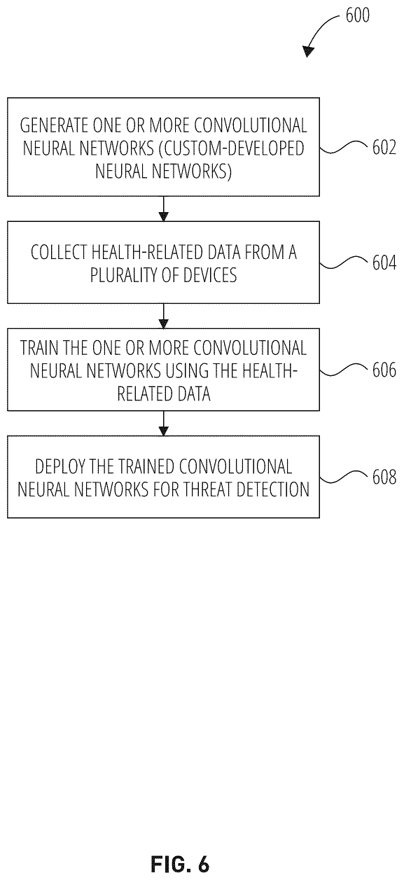

FIG. 6 illustrates a method of developing and training a convolutional neural network for threat detection according to some aspects of the present disclosure.

Method 600 may likewise be implemented by architecture 100 of FIG. 1 and software architecture 200 of FIG. 2, for example by processors 112, 114 executing CNN-based models 206 using health-related data stored in databases 108, 110.

At step 602, one or more convolutional neural networks (CNNs) are generated. In some aspects, the CNNs are customized one-dimensional convolutional neural network models configured to operate directly on raw time-series health-related data. The customized CNNs may include a sequence of one-dimensional convolutional layers with 1-dimensional convolutional kernels (e.g., kernels of size 13 samples) followed by 1-dimensional max pooling layers that downsample by a factor of four in the time dimension. After each max pooling layer, the number of channels (feature maps) in the subsequent convolutional layers may be doubled so that the CNNs progressively learn more complex representations as the temporal resolution decreases. In one example, a first convolutional layer increases the number of channels to 10 and subsequent convolutional layers increase the number of channels up to 320. In some aspects, all convolutional layers except a last convolutional layer are followed by max pooling layers; the last convolutional layer may instead be followed by an average pooling layer that aggregates temporal information prior to one or more fully connected layers used for classification. A first convolutional layer may use a stride of 5 to enlarge the receptive field of the network and to enable the customized CNNs to capture long-range temporal patterns that correlate with threat exposure.

At step 604, health-related data is collected from a plurality of devices. Similar to 502 of method 500, the health-related data may be obtained from wearable devices (e.g., devices 102, 104, 106) and may include heart rate, respiration rate, sleep data, and/or other physiological measurements collected at various sampling intervals. The health-related data may be preprocessed to a format suitable for CNN input, such as normalized time windows of fixed duration (e.g., several days of data prior to a test event), and may optionally be resampled to a common sampling rate across features using interpolation or other resampling techniques. In some aspects, different CNNs may be defined per feature type (e.g., a heart-rate CNN and a respiration-rate CNN), and multimodal fusion techniques such as late fusion may be applied by combining model outputs as described with reference to FIG. 3.

At step 606, the one or more customized CNNs generated at 602 are trained using the health-related data collected at 604. Training may be performed using the AdamW optimizer with a learning rate of about 0.001 and a weight decay of about 0.01, and may leverage mixed-precision training to increase computational efficiency. The CNNs may be trained using labeled examples indicating positive and negative threat exposure, and the training may be organized into epochs over randomized mini-batches of samples. During training, hyperparameters such as depth, width, kernel sizes, stride values, dilation parameters, and learning rate schedules may be tuned via grid search and/or manual experimentation to optimize predictive performance and to prevent underfitting or overfitting. In some aspects, the receptive field of the CNNs (determined by kernel sizes, strides, and pooling operations) may be selected based on the sampling rate of the underlying time-series data so that the CNNs can learn patterns spanning time intervals relevant to disease progression. The trained CNNs may output, for a given input window of time-series data, a probability or score representing likelihood of threat exposure.

At step 608, the trained convolutional neural networks are deployed for threat detection. For example, the trained CNN models may be stored in databases 108, 110 and loaded on processors 112, 114 for on-line inference. Incoming streams of health-related data from the plurality of devices may be segmented into windows and passed through the deployed CNNs, which generate threat scores and/or classifications in near real time. In instances where multiple CNNs are used (e.g., separate CNNs for different modalities), a fusion module may combine their outputs, such as by averaging post-SoftMax probabilities, to produce a final decision. In some aspects, the deployed CNNs may operate in conjunction with the ensemble models of method 500, for example as part of an ensemble-of-ensembles or hybrid decision engine. Method 600 may be repeated to retrain and update the customized CNNs as additional data is collected, changes in threat profiles occur, or as improved architectures are developed.

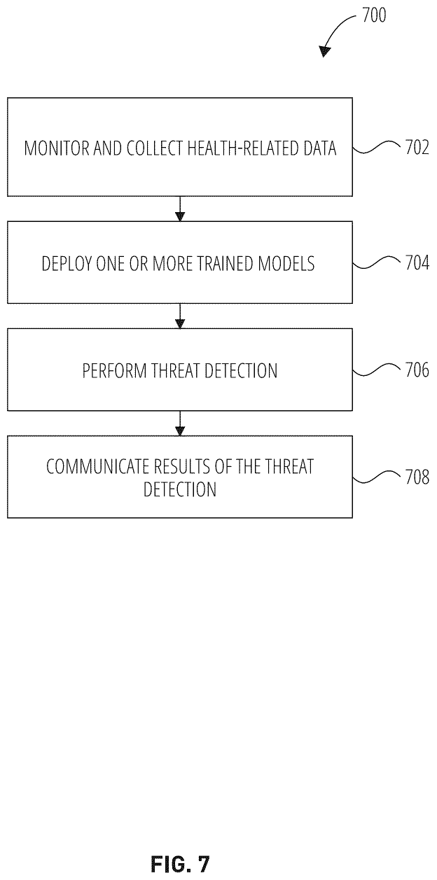

FIG. 7 illustrates a method of threat detection using one or more trained machine learning models according to some aspects of the present disclosure.

Method 700 may be performed using any combination of the trained ensemble models produced by method 500 and the trained CNN models produced by method 600, and may be executed by architecture 100 of FIG. 1 in an operational deployment of BiCEPS.

At step 702, health-related data is monitored and collected. In some aspects, this operation corresponds to continuous or periodic monitoring of physiological signals from a plurality of devices, such as wearable devices 102, 104, 106, for a plurality of users. The devices may locally sense, buffer, and/or pre-aggregate sensor readings, and transmit the health-related data to databases 108, 110 over wired and/or wireless communication channels. The monitoring at 702 may occur in substantially real time and may span extended time windows (e.g., days or weeks) to capture both baseline physiology and changes associated with potential exposure to biological and/or chemical threats. The collected health-related data may optionally undergo light preprocessing (e.g., normalization, resampling, artifact removal) to match the data formats expected by the trained models.

At step 704, one or more trained models are deployed for use with the monitored health-related data. In one example, a model-selection and orchestration component loads the trained ensemble models and/or customized CNN models from model repositories in databases 108, 110 and instantiates them on processors 112, 114 or other computing resources (e.g., cloud-based compute nodes). Different models may be designated for different device types, feature sets, or operating scenarios (e.g., asymptomatic screening versus symptomatic monitoring). The deployment at 704 may include configuring thresholds, decision rules, and fusion strategies (e.g., late fusion across modalities or across model families) to obtain desired trade-offs between sensitivity, specificity, false positive rate, and computational efficiency.

At step 706, threat detection is performed using the deployed trained models and the monitored health-related data. For example, the health-related data collected at 702 may be partitioned into analysis windows (e.g., fixed-length sequences) and provided as input to one or more of the trained ensemble models and/or CNN models. The models produce scores and/or classification outputs indicating likelihood of exposure to one or more biological and/or chemical threats. In some aspects, the threat detection may be configured to identify exposure before onset of associated symptoms by focusing on temporal patterns that emerge in physiological signals prior to clinically observable events. The threat detection at 706 may additionally account for confounding factors (e.g., physical activity, sleep disruption) that could otherwise mimic threat-related changes in the health-related data, for example by incorporating such factors into the features used by the models or by using specialized models trained to differentiate between threat-related and non-threat-related patterns.

At step 708, results of the threat detection are communicated. In some aspects, communication at 708 may include generating alerts, notifications, dashboards, or reports based on the outputs of the trained models. For example, when a threat score exceeds a configurable threshold, a notification may be transmitted to an affected user, a healthcare provider, a public health authority, and/or another monitoring entity. The notification can indicate that the user has been identified as likely exposed to a biological and/or chemical threat, optionally specifying a confidence level, time window of concern, and recommended follow-up actions (e.g., confirmatory testing, isolation, additional monitoring). Results may additionally be logged and stored in databases 108, 110 for auditing, further analysis, or use in future retraining of the models. In some aspects, communication at 708 may also include aggregating anonymized threat detection results across many users to provide population-level situational awareness and to support early detection and containment of outbreaks.

Although method 700 is illustrated with discrete steps 702-708, the operations may be implemented in various orders and can be performed continuously and/or iteratively. For example, monitoring and collecting health-related data at 702 may occur concurrently with performing threat detection at 706, and the deployment of models at 704 may be dynamically updated as new or improved trained models become available.

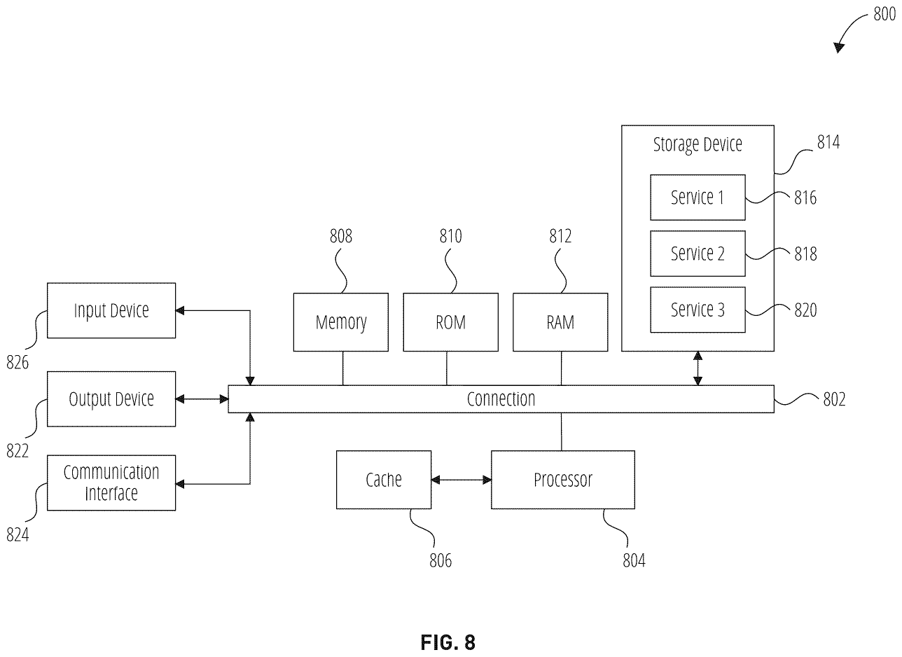

FIG. 8 shows an example of computing system that can be used as any one or more components of BiCEPs according to some aspects of the present disclosure.

Computing system 800 can be any one of the components of architecture 100 of FIG. 1. Various components of computing system 800 may be in communication with each other using connection 802. Connection 802 can be a physical connection via a bus, or a direct connection into processor 804, such as in a chipset architecture. Connection 802 can also be a virtual connection, networked connection, or logical connection.

In some examples, computing system 800 is a distributed system in which the functions described in this disclosure can be distributed within a datacenter, multiple data centers, a peer network, etc. In some embodiments, one or more of the described system components represents many such components each performing some or all of the function for which the component is described. In some embodiments, the components can be physical or virtual devices.

Example computing system 800 includes at least one processing unit (CPU or processor) 804 and connection 802 that couples various system components including system memory 808, such as read-only memory (ROM) 810 and random access memory (RAM) 812 to processor 804. Computing system 800 can include a cache of high-speed memory 806 connected directly with, in close proximity to, or integrated as part of processor 804.

Processor 804 can include any general purpose processor and a hardware service or software service, such as services 816, 818, and 820 stored in storage device 814, configured to control processor 804 as well as a special-purpose processor where software instructions are incorporated into the actual processor design. Processor 804 may essentially be a completely self-contained computing system, containing multiple cores or processors, a bus, memory controller, cache, etc. A multi-core processor may be symmetric or asymmetric.

To enable user interaction, computing system 800 includes an input device 826, which can represent any number of input mechanisms, such as a microphone for speech, a touch-sensitive screen for gesture or graphical input, keyboard, mouse, motion input, speech, etc. Computing system 800 can also include output device 822, which can be one or more of a number of output mechanisms known to those of skill in the art. In some instances, multimodal systems can enable a user to provide multiple types of input/output to communicate with computing system 800. Computing system 800 can include communication interface 824, which can generally govern and manage the user input and system output. There is no restriction on operating on any particular hardware arrangement, and therefore the basic features here may easily be substituted for improved hardware or firmware arrangements as they are developed.

Storage device 814 can be a non-volatile memory device and can be a hard disk or other types of computer readable media which can store data that are accessible by a computer, such as magnetic cassettes, flash memory cards, solid state memory devices, digital versatile disks, cartridges, random access memories (RAMs), read-only memory (ROM), and/or some combination of these devices.

The storage device 814 can include software services, servers, services, etc., that when the code that defines such software is executed by the processor 804, it causes the system to perform a function. In some embodiments, a hardware service that performs a particular function can include the software component stored in a computer-readable medium in connection with the necessary hardware components, such as processor 804, connection 802, output device 822, etc., to carry out the function.

For clarity of explanation, in some instances, the present technology may be presented as including individual functional blocks including functional blocks comprising devices, device components, steps or routines in a method embodied in software, or combinations of hardware and software.

Any of the steps, operations, functions, or processes described herein may be performed or implemented by a combination of hardware and software services or services, alone or in combination with other devices. In some examples, a service can be software that resides in memory of a client device and/or one or more servers of a content management system and perform one or more functions when a processor executes the software associated with the service. In some examples, a service is a program or a collection of programs that carry out a specific function. In some embodiments, a service can be considered a server. The memory can be a non-transitory computer-readable medium.

In some examples, the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer-readable media. Such instructions can comprise, For example, instructions and data which cause or otherwise configure a general purpose computer, special purpose computer, or special purpose processing device to perform a certain function or group of functions. Portions of computer resources used can be accessible over a network. The executable computer instructions may be, For example, binaries, intermediate format instructions such as assembly language, firmware, or source code. Examples of computer-readable media that may be used to store instructions, information used, and/or information created during methods according to described examples include magnetic or optical disks, solid-state memory devices, flash memory, USB devices provided with non-volatile memory, networked storage devices, and so on.

Devices implementing methods according to these disclosures can comprise hardware, firmware and/or software, and can take any of a variety of form factors. Typical examples of such form factors include servers, laptops, smartphones, small form factor personal computers, personal digital assistants, and so on. The functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

Although a variety of examples and other information was used to explain aspects within the scope of the appended claims, no limitation of the claims should be implied based on particular features or arrangements in such examples, as one of ordinary skill would be able to use these examples to derive a wide variety of implementations. Further and although some subject matter may have been described in language specific to examples of structural features and/or method steps, it is to be understood that the subject matter defined in the appended claims is not necessarily limited to these described features or acts. For example, such functionality can be distributed differently or performed in components other than those identified herein. Rather, the described features and steps are disclosed as examples of components of systems and methods within the scope of the appended claims.

Claim language or other language reciting “at least one of” a set and/or “one or more” of a set indicates that one member of the set or multiple members of the set (in any combination) satisfy the claim. For example, claim language reciting “at least one of A and B” or “at least one of A or B” means A, B, or A and B. In another example, claim language reciting “at least one of A, B, and C” or “at least one of A, B, or C” means A, B, C, or A and B, or A and C, or B and C, or A and B and C. The language “at least one of” a set and/or “one or more” of a set does not limit the set to the items listed in the set. For example, claim language reciting “at least one of A and B” or “at least one of A or B” can mean A, B, or A and B, and can additionally include items not listed in the set of A and B.

What is claimed is:

1. A method comprising:
collecting health-related data from a plurality of devices;
performing feature extraction on the health-related data, wherein the feature extraction includes:
identifying statistical and temporal features of the health-related data;
applying linear regression to the statistical and temporal features to determine one or more trends in the statistical and temporal features;
applying one or more rolling windows to the one or more trends to identify short-term and long-term variations in the statistical and temporal features; and
generating lagged values of the health-related data to determine past behavior of the statistical and temporal features; and
training one or more machine learning models using the statistical and temporal features, the one or more trends, the short-term and long-term variations, and the past behavior of the statistical and temporal features.

2. The method of claim 1, wherein the statistical and temporal features include one or more of a mean, a median, a standard deviation, a minimum, a maximum, a skew, and a kurtosis.

3. The method of claim 1, wherein the one or more trends include increasing and decreasing trends.

4. The method of claim 1, wherein the one or more machine learning models include one or more of Gradient Boost, Extreme Gradient Boost, Random Forest, and AdaBoost.

5. The method of claim 1, further comprising:
deploying the one or more machine learning models once the is complete; and
performing threat detection using the one or more machine learning models.

6. The method of claim 5, wherein the threat detection includes detecting one or more of a biological threat and a chemical threat.

7. The method of claim 6, wherein the threat detection includes identifying exposure to one or more of the biological threat and the chemical threat before onset of associated symptoms.

8. The method of claim 1, wherein the health-related data includes heart rate, respiration rate, and sleep category data.

9. The method of claim 1, wherein the health-related data is collected for a plurality of users.

10. The method of claim 1, wherein the plurality of devices are wearable devices.

11. A method comprising:
generating a customized convolutional neural network model;
collecting health-related data from a plurality of devices; and
training the customized convolutional neural network model using the health-related data.

12. The method of claim 11, wherein the customized convolutional neural network model is a 1-dimensional neural network with 1-dimensional convolutional kernels.

13. The method of claim 12, wherein convolutional layers of the customized convolutional neural network model are followed by 1-dimensional max pooling layers.

14. The method of claim 13, wherein the 1-dimensional max pooling layers are down sampled by a factor of 4 in one dimension.

15. The method of claim 13, wherein after each of the 1-dimensional max pooling layers, a number of channels for the convolutional layers is doubled.

16. The method of claim 13, wherein the convolutional layers exclude a last convolutional layer of the customized convolutional neural network model.

17. The method of claim 16, wherein the last convolutional layer is followed by an average pooling layer.

18. The method of claim 11, wherein the customized convolutional neural network model is trained using AdamW optimizer with a learning rate of 0.001 and a weight decay of 0.01.

19. The method of claim 11, wherein the customized convolutional neural network model is trained using PyTorch mixed precision training.

20. The method of claim 13, wherein a first convolutional layer of the convolutional layers uses a stride of 5 to increase a size of a receptive field for identifying long-range patterns in the health-related data.
